(19) United States
(12) Patent Application Publication
(10) Pub. No.: US 2020/0121190 A1
(43) Pub. Date: Apr. 23, 2020

(54) DEVICES, SYSTEMS AND METHODS RELATING TO HAND-HELD COMMUNICATIONS DEVICES FOR IN SITU DIFFERENTIATION BETWEEN VIRAL AND NON-VIRAL INFECTIONS

(71) Applicant: YES Biotechnology Inc., Vancouver (CA)

(72) Inventor: Peter Whitehead, Vancouver (CA)

(73) Assignee: YES Biotechology Inc., Vancouver, BC (CA)

(21) Appl. No.: 16/612,033

(22) PCT Filed: May 9, 2018
(86) PCT No.: PCT/CA2018/050555

§ 371 Date: Nov. 8, 2019

Related U.S. Application Data
(60) Provisional application No. 62/503,816, filed on May 9, 2017.

(51) Int. Cl. A61B 5/00; A61B 5/01; G16H 30/40; G16H 50/20

(52) U.S. Cl. CPC A61B 5/0071; A61B 5/01; A61B 5/0077; G16H 40/63; G16H 30/40; G16H 50/20; A61B 5/6898

(57) ABSTRACT

Detection systems and methods configured to scan and interpret a suspected infection at in vivo biological target site, comprising emitting excitation light selected to elicit fluorescent light from a suspected infection at the target site; sensing fluorescent light emanating from the target site elicited by such excitation light; sensing heat levels above ambient body temperature emanating from the target site; and then based at least in part on the sensed fluorescent light and the heat levels, determining a probability whether the target site comprises an infection.

### BACKGROUND

Detection and determination of and between biological infections such as bacterial and viral infections have always been difficult and uncertain processes. The importance of accurate detection and determination has increased with the advent of antibiotic-resistant strains of bacteria such as Methicillin-resistant Staphylococcus aureus (MRSA), which some people have attributed to the over-prescription of antibiotics for virtually all forms of infections including patients with sore throats even if those infections are viral and thus not improved by antibiotics.

Accordingly, there has gone unmet a need to improve the ability of a doctor, nurse, dentist or other person or user to detect and diagnose infections as viral or non-viral, typically bacterial.

The present systems and methods, etc., provide improved abilities to detect and diagnose infections as viral or non-viral, typically bacterial, using heat and light sensing technologies implemented via a mobile communications device such as a cell phone, and/or other advantages.

### SUMMARY

The present systems, devices and methods, etc., relate to in situ photonic and thermic detection systems using heat and light sensing technologies to detect and diagnose infections as viral or non-viral, typically bacterial, the systems sized and configured to be attached to mobile communications devices such as a cell phone. Methods and systems related to such detection and diagnosis or identification are discussed and shown in U.S. patent application Ser. No. 15/350,626, filed Nov. 14, 2016 and entitled Devices, Systems And Methods Relating To In Situ Differentiation Between Viral And Bacterial Infections; a copy of such application is appended to the end of this provisional application.

In one aspect of the current application, the systems and methods relating to in situ differentiation are implemented via a mobile communications housing sized and configured for detection of a suspected infection at a target site, the mobile communications housing sized and configured for attachment to a mobile communication device, the mobile communications housing comprising a detection system comprising a) an excitation light source configured to emit excitation light selected to elicit fluorescent light from the suspected infection at the target site, b) a light sensor configured to detect the fluorescent light, c) a heat sensor configured to detect and identify thermal data indicating heat above ambient body temperature emanating from the suspected infection at the target site, and d) at least one of a mobile communications housing camera configured to accept substantially only fluorescent light emanating from the target site or a camera port sized and configured to selectively transmit substantially only fluorescent light emanating from the target site to an interior camera disposed within the mobile communication device, and, the mobile communications housing further operably connected computer-implemented programming configured to a) accept fluorescent light data associated with the fluorescent light and thermal data associated with the heat levels above ambient body temperature, and b) interpret the data to determine a probability whether the target site contains an infection.

In some embodiments, the mobile communications housing further comprises a power source operably connected to power the excitation light source, and can comprise a computer containing the computer-implemented programming, and wherein the power source is operably connected to power the computer. The computer-implemented programming can also or instead comprise an app run by a computer disposed within the mobile communication device. The excitation light source can comprise a light emitting diode configured to emit substantially only the excitation light, and can emit substantially only a single wavelength or wavelength band of excitation light and/or can comprise multiple excitation light emitters each emitting a different wavelength or wavelength band of excitation light. The excitation light source can comprise a white light emitter and at least one short pass filter configured to selectively transmit substantially only light below about 485 nm. The excitation light source can comprise a light port comprising at least one short pass filter configured to selectively transmit substantially only wavelengths below about 485 nm emitted from a light source disposed within mobile communications device.

The mobile communications housing camera or camera port can comprise at least a first long pass filter configured to block the excitation light and a notch filter configured to selectively transmit substantially only fluorescent light emanating from the target area. The long pass filter can comprise an about 475 nm long pass filter, and the notch filter transmits light have a wavelength of about 590 nm. The mobile communications housing camera or camera port can comprise at least one filter configured to selectively transmit substantially only two wavelength bands from about 475-585 nm and at about 595 nm. The mobile communications housing camera or camera port can be configured to selectively accept or transmit, respectively, at least a) substantially only fluorescent light emanating from the target area, or b) all visible light wavelengths emanating from the target area.

The system is suitable for detecting and differentiating between viral and non-viral/bacterial infections in an animal body, such as in the throat, on the skin, or in the mouth, gut, vagina, lungs or other location capable of hosting such infections. In one aspect, the system contains an appropriate sensor (CCD, CMOS, thermopiles, etc.) configured to capture at least two groups of data, one corresponding to emitted fluorescence wavelengths, typically autofluorescence, from a suspected viral or non-viral infection, for example such as bacteria, and one for capturing a heat signature caused by such non-viral agent—or not present in the case of a viral infection. Exemplary excitation wavelengths include about 340 nm and 380 nm-500 nm, and detection wavelengths include 500 nm to 700 nm for fluorescence signatures and 700 nm+ for heat signatures (thermal data) when heat is being detected using IR (infrared). The thermal infrared region for room temperature objects is generally considered to be about 1000-1500 nm depending on which technology is being used to measure it. Suitable thermopiles for use herein can look at window of about 800-1400 nm. Other methods of heat/thermal data detection or measurement can also be employed such as measurement of heat conduction or convection, which can in some instances be measured using a contact measurement device such as a contact thermometer. Exemplary temperature levels include any substantial increase over ambient body temperature for the patient/organism commensurate with heat generated by bacteria, for example increases of about 0.5° C., 1° C., 2° C., or 3° C.

The fluorescence can come from fluorophores contained in or caused by the target bacteria such as porphyrins or can be introduced into to the target area if desired, for example as fluorophores that have been immuno-tagged to be species-specific or that are egested by specific species. Further, in the event of a viral infection, the autofluorescent signature of the native, ambient tissue is reduced or eliminated, and thus the loss of native autofluorescence is an indicator of a viral infection. If desired, the system can also detect other wavelengths or wavelengths bands of light such as white light, all visible light, or selectively blue light or red light, or selectively IR (infrared) etc. Such systems can also provide photographs or video, including real-time or live photographs or video.

The systems can also comprise light sources suitable to provide interrogative light for the examination of the target area. Such light sources can include, for example, a broad spectrum light source with appropriate selective light filters to pass only desired wavelengths such as blue wavelengths suitable for exciting autofluorescence, infrared wavelengths suitable for heating the target area, as well as visible-light imaging wavelengths such as red-green-blue (rgb) or cyan-yellow-magenta (cym) wavelengths. The light source can also comprise a plurality of different light sources each tasked with providing a desired set(s) of wavelengths or a wavelength range(s); such sources can also be used in combination if desired. Examples of such sources include LED, metal halide, and xenon light sources.

The detected fluorescence and heat-based radiation provide a set(s) of captured data. The captured data can be viewed in real-time by a user and/or can be sent to a desired location. For example, the data can be sent as a file or set of files preferably with an image representing the target site, to a computer such as desktop computer, laptop computer, an iPad® or PDA, where the data is processed and/or can be viewed by human interrogators. The processed data can be interpreted by the user and/or a computer to identify the type of target organism (e.g., whether it is a virus or bacterium). Such information can be useful for determining appropriate treatment options—or non-treatment options such as choosing not to use antibiotics against a viral infection.

In some embodiments, the processed data/image can provide a score of the combined data points based on infrared hypothermic and/or hyperthermic values and can also incorporate or provide a spatial organization of aggregated amounts of abnormal thermal and fluorescent conditions within the target area. Generally speaking, a lack of thermic activity above ambient body temperature indicates that an infection is viral, whereas presence of substantial thermic activity above ambient body temperature indicates the infection is bacterial. Such spatial organization can be provided to the practitioner to improve the ability to visualize the affected area, and can also be incorporated in the diagnosis aspect of the systems herein as spatial organization, such as presence, color and shape of bacterial colonies, can be indicative of different types of infections.

In other words, in some embodiments the devices, etc., herein can distinguish between bacterial and viral infections and if desired can also help determine the location of the infection(s) within a target area. For the example of a patient arriving at a clinic (or other provider) with a sore throat, the processed information can indicate to the caregiver a probability, such as more than about 50%, 80%, 90%, 95%, 98%, 99% or 100%, that the sore throat is an infection and if so, whether it is a bacterial infection or viral infection, as well as, if desired, location(s) in the throat of the infections.

The devices can rely on auto-generated radiation such as autofluorescence generated autonomously within the infecting organism or a heat signature (or lack thereof in the case of viruses), or the devices can emit fluorescence-inducing light and/or heat-inducing light if desired.

In some aspects, the current application is directed to detection systems configured to scan and interpret a suspected infection at in vivo biological target site, the detection system comprising a mobile communications housing comprising at least one light emitter configured to emit excitation light selected to elicit fluorescent light from the suspected infection at the target site, a light sensor configured to detect the fluorescent light, and a heat sensor configured to detect and identify thermal data indicating heat above ambient body temperature emanating from the suspected infection at the target site, the detection system further operably connected computer-implemented programming configured to a) accept fluorescent light data associated with the fluorescent light and thermal data associated with the heat levels above ambient body temperature, and b) interpret the data to determine a probability whether the target site contains an infection.

The system can be further configured to determine whether the suspected infection can be a viral infection or a non-viral infection, can further comprise an imaging system aimed and configured to provide an image of the target site. The image of the target site can identifies a spatial organization of the suspected infection and the system can utilizes such spatial organization when determining the probability whether the infection can be a viral infection or a non-viral infection and/or when determining an identity of an infectious agent in the suspected infection. When the suspected infection is a non-viral infection, the computer implemented programming can further identify whether the infection may be bacterial.

The at least one light emitter, the light sensor and the heat sensor can be all located at a distal end of the mobile communications housing and can be all forward-facing and aimed to substantially cover a same area of the target site. The mobile communications housing can be configured to be held in a single hand of a user and can be configured to fit within a human oral cavity and to scan at least a rear surface of such oral cavity or a throat behind such oral cavity.

The system further can comprise a separable distal element sized and configured to removably attach to the distal end of the mobile communications housing, wherein the separable distal element comprises at least one of light-blocking sides and/or a forward-facing window configured to selectively transmit at least the excitation light, the fluorescent light and the heat levels without substantial alteration. If desired, at least two sides of the separable distal element comprise recesses configured to keep the sides out of a view of the heat sensor. The distal end of the mobile communications housing and the separable distal element can be cooperatively configured such that the separable distal element can be snapped on and off the distal end of the mobile communications housing, for example via cooperative projections and detents configured such that the separable distal element can be snapped on and off the distal end of the mobile communications housing.

The distal end of the mobile communications housing can be configured to be mounted onto a single circuit board when the mobile communications housing can be not being used for scanning, and can further comprise a display screen on a dorsal side of the mobile communications housing.

The system can be configured to account for heat level distortions due to ambient conditions at the target site, for example using specific anti-distortion structures and/or by using at least one algorithm configured to account for the heat level distortions.

In further aspects, the current application is directed methods of scanning in vivo biological target site for a suspected infection, the methods comprising:
emitting excitation light selected to elicit fluorescent light from a suspected infection at the target site
sensing fluorescent light emanating from the target site elicited by such excitation light;
sensing thermal data indicating heat above ambient body temperature emanating from the target site
based at least in part on the sensed fluorescent light and the heat levels, determining a probability whether the target site comprises an infection.

Such methods can comprise, utilize or implement the structures and devices discussed herein. Such methods can also comprise making such structures and devices discussed herein

These and other aspects, features and embodiments are set forth within this application, including the following Detailed Description and included drawings. Unless expressly stated otherwise, all embodiments, aspects, features, etc., can be mixed and matched, combined and permuted in any desired manner In addition, various references are set forth herein, including but not limited to the Cross-Reference To Related Applications, that discuss certain systems, apparatus, methods and other information; all such references are incorporated herein by reference in their entirety and for all their teachings and disclosures, regardless of where the references may appear in this application.

### BRIEF DESCRIPTION OF THE DRAWINGS

FIG. 1 depicts a perspective view of exemplary, stylized depictions of a mobile communications device and mobile communications housing as discussed herein.

FIG. 2 depicts a perspective view of an exemplary, stylized depiction of a mobile communications device attached to a mobile communications housing as discussed herein.

FIG. 3 depicts side view and front plan view of an exemplary, stylized depiction of a mobile communications device attached to a mobile communications housing in use to examine a target site as discussed herein.

FIG. 4 depicts a rear plan view of an exemplary, stylized depiction of a mobile communications device as discussed herein without a supplemental mobile communications housing.

FIG. 5 depicts a flow chart of an exemplary system software lifecyle.

FIG. 6 depicts an exemplary embedded software architecture and its composition of individual software components.

FIG. 7 depicts a flow chart of an exemplary application executive state diagram.

### DETAILED DESCRIPTION

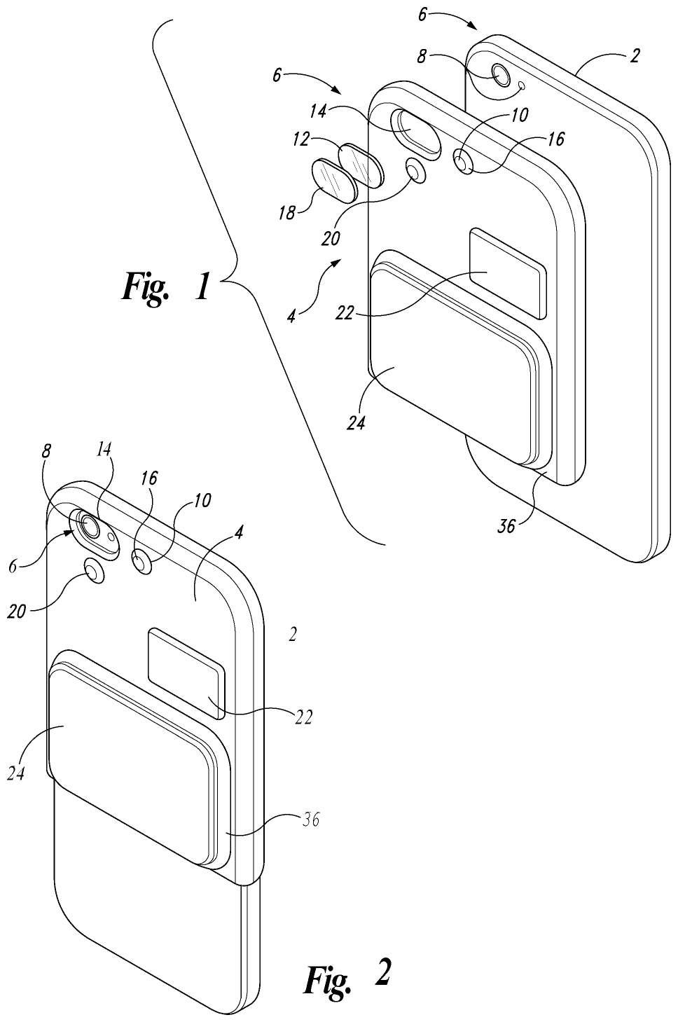

Turning to the Figures, FIGS. 1 and 2 depict an exemplary, stylized depiction of a mobile communications device 2 (such as a smart phone or other handheld device comprising both imaging (e.g., via camera 8) and data transmission capabilities), and also depicting a mobile communications housing 4 attachable to a mobile communications device 2 discussed herein; in FIG. 2, the mobile communications housing 4 is attached to the mobile communications device 2. In the embodiments shown, the mobile communications housing 4 includes an excitation light source 16, in this instance an LED disposed on the mobile communications housing 4. The excitation light source 16 is configured to emit excitation light selected to elicit fluorescent light from the suspected infection at the target site. The mobile communications housing 4 also includes a camera port 14 comprising both a dichroic filter 18 and a notch filter 12. The camera port 14 is sized and configured to selectively transmit or admit substantially only fluorescent light emanating from the target site to an interior camera 8 disposed within the mobile communication device. The mobile communications housing 4 further includes a heat sensor configured to detect and identify thermal data indicating heat above ambient body temperature emanating from the suspected infection at the target site.

The mobile communications housing 4 further comprises a power source operably connected to power the excitation light source 16, as well as a computer 36 containing the computer-implemented programming, and the computer 36 also operably connected to the power source such as battery 24. The mobile communications housing 4 further contains a wireless communications unit such as Bluetooth® communications unit 22 to transmit data and diagnoses and other information to/from the mobile communications housing 4 and the mobile communications device 2, and to other operably connected devices such as printers, additional computers, viewing screens, etc., if desired.

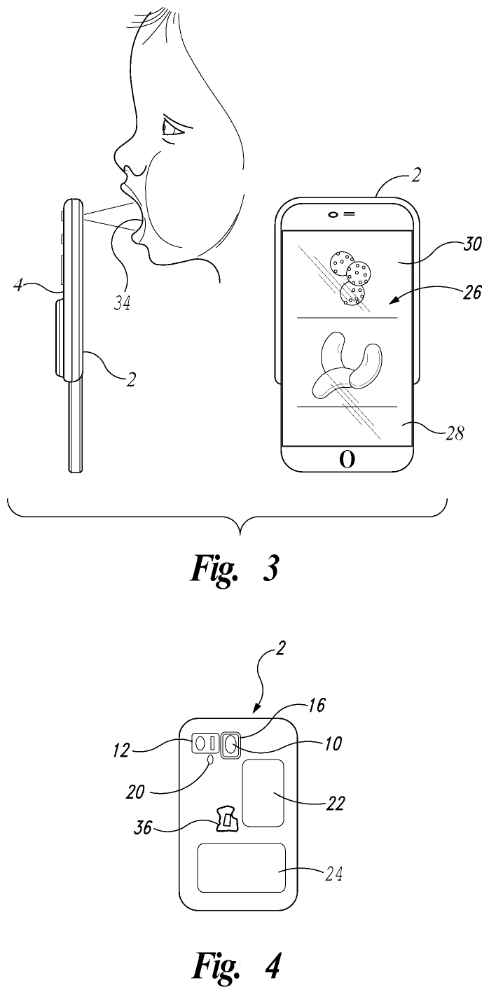

FIG. 3 depicts an exemplary, stylized depiction of the mobile communications housings 4 and mobile communications devices 2 discussed herein in use, with excitation light 32 shining from the mobile communications housing 4 and mobile communications device 2 onto a target site 34. An image 26 and diagnostic information 28 is shown on the screen 30 of the mobile communications device 2.

FIG. 4 depicts an exemplary, stylized depiction of a mobile communications device 2 version as discussed herein, without an attached mobile communications housing 4 providing certain of the hardware discussed herein. In other words, in this embodiment the various features and structures used for the systems herein are all maintained within the mobile communications device 2 without need for an added-on mobile communications housing 4.

Turning to a general discussion of exemplary detection and diagnostic aspects and embodiments of the systems herein, such discussions are augmented by, and hereby include, the discussions set forth in the appended copy of U.S. patent application Ser. No. 15/350,626. The illumination and detection aspects of the systems herein emit the selected interrogation wavelengths (for example via distally carried LED light emitters or via proximally located light sources where such light is conducted through appropriate conductors such as optic fibers to the target site) and then to carry the elicited photonic data (fluorescence data) and heat data/thermal data (photonic or otherwise) gathered from the interrogation site to the user such as a doctor or other health care provider. The scope can if desired include elements to conduct an optical image directly from the target site to the viewer/user. The system can also include computers and the like, for example located proximally via hardwire or wireless links or within the interrogative device, to process the data and if desired provide estimates of the presence or absence of bacteria at the interrogation/target site, and estimates of whether the suspected infection, if present, is or is not viral.

The device can be sized and configured to be held by a human hand, i.e., is a “hand-held”, for certain embodiments and can be a device shaped to be maintained outside the body as shown, for example, in US patent application no. 20050234526, or can be a catheter or endoscope or other configuration (e.g., colposcope, laparascope, etc.) shaped to be inserted into or otherwise introduced into or aimed toward the body of a patient.

The scope, for example where the scope provides an image to an ocular, can comprise a hollow casing with desired optics that returns light from the target tissue to the detector and/or an ocular eye piece. The hollow casing if desired can also transmit light from an external (typically proximally-located) light source to the target tissue. Suitable ocular eye pieces include an eye cup or frosted glass, and can be monocular or binocular as desired. If desired, the scope can alternatively, or additionally, be configured to contain one or more internal light sources, distally located light sources (such as LEDs), and/or proximally located light sources, and one or more fiber optic light guides, fiber optic cables or other such light transmission guides, in addition to, or instead of, the light guide formed by the hollow casing discussed above.

Typically, the scope comprises a power source suitable to power the light sources and/or sensors, data transmitters, and other electronics associated with the device. The power source can be an external power source such as a battery pack connected by a wire, a battery pack maintained in the handle or otherwise within the scope itself, or a cord and plug or other appropriate structure linking the device to a wall outlet or other power source. In some embodiments, the mobile communications housing 4 of the light source includes a retaining structure configured to hold the scope to a desired location when not in use.

As noted previously, the scope comprises one or more sensors such as CCDs, CIDs, CMOSs, thermopiles, etc., and/or is operably connected to one or more display devices, which can be located on the scope and/or in an operably connected computer. Such sensors, either in combination or as wide-sensing singular sensors, can detect at least any desired fluorescence, such as autofluorescence in the 400 nm-600 nm range and 700 nm+ range. Suitable sensors including infrared (IR) and detectors are well known.

Exemplary display devices include CRTs, flat panel displays, computer screens, etc. The diagnostic systems include one or more computers that control, process, and/or interpret the data sets and if desired various other functions of the scope, including, for example, diagnostic, investigative and/or therapeutic functions. Typically, a computer comprises a central processing unit (CPU) or other logic-implementation device, for example a stand-alone computer such as a desk top or laptop computer, a computer with peripherals, a handheld, a local or internet network, etc. Computers are well known and selection of a desirable computer for a particular aspect or feature is within the scope of a skilled person in view of the present disclosure.

As noted above, suitable heat detectors include well known infrared (IR) and including for example thermopiles and microbolometer arrays, provided that when such devices are included within the scopes/housings herein, such are suitably sized to fit within or on the scope without making the overall device too large for its purpose. Where the detection light gathered from the target sight is transported, such as by fiber optics, outside the scope and body, size concerns for the heat detector elements (and other detection elements) are reduced. Such sensors can also comprise heat-neutralization structures configured to reduce or eliminate improper ambient heat readings due to outside influences, such as a patient's breath when interrogating the back of the mouth or throat. Heat-neutralization structures can include, for example, an anti-fog element such as a hydrophobic material, a spray or coating that does not skew the signal determined by the sensor, or a dichroic mirror that transmits the signal to a proximate sensor removed from the impeding outside influence.

### EXAMPLES

### Example 1: Exemplary Software Design

An exemplary system comprises embedded system software and host client software. The embedded system software will run on a Raspberry PI (RPI) Compute Module. This software can comprise device drivers, kernel services, the Linux kernel and bootloader, and application level software. The host software is a client Graphical User Interface (GUI) that will run on a PC. The client GUI aids users in interacting with the system.

Table 1 in FIG. 5 shows an exemplary system level software lifecycle for system during a typical use case scenario. Aspects of the system functionality can be encapsulated within the “Application Executive” sub-process.

In FIG. 5, the exemplary software lifecycle comprises power on 500 followed by bootloader 502, which in turn leads to splash screen 504. The splash screen 504 is followed by kernel boot startup scripts 506, which then invokes the application executive 508. At the end of the cycle, power-off 510 takes place.

Embedded System Software

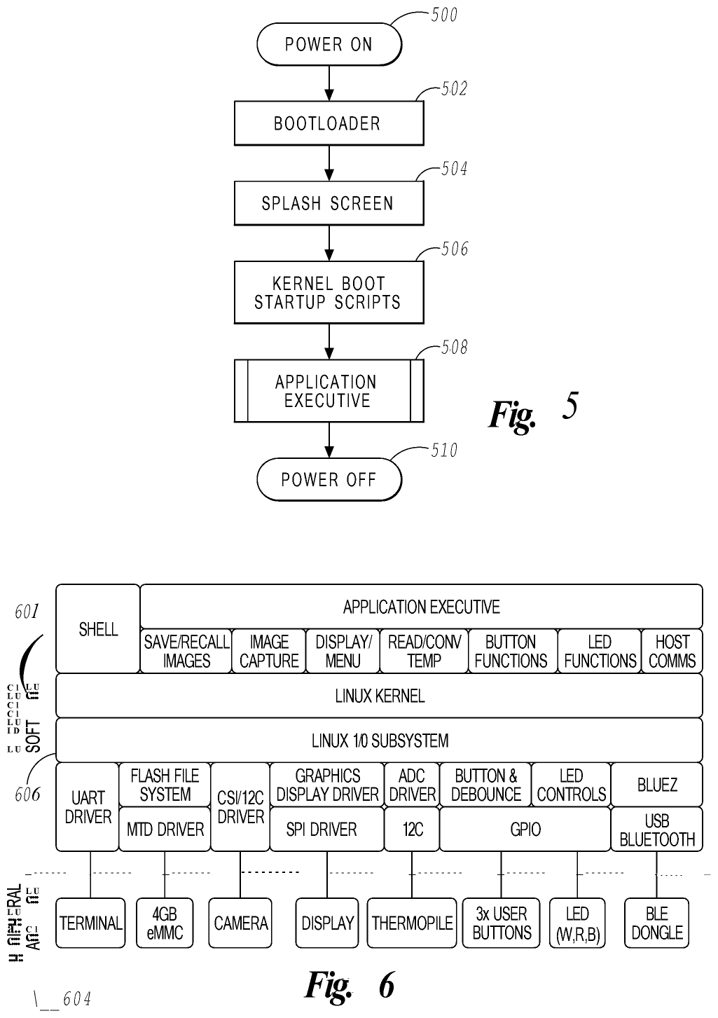

Turning to FIG. 6, the embedded hardware platform 602 can comprise a RPI Compute Module with a number of hardware peripherals 604 that make use of the Compute Module's Input/Output (I/O) 606. The compute module utilizes a Broadcom BCM2835 processor with on-board 512 MB of RAM and 4 GB of eMMC flash. Additionally, the Compute Module pulls out all of the I/O pins of the processor for developer use. The Compute Module has a rich embedded Linux ecosystem making it ideally suited for rapid prototyping and deployment of embedded Linux. The embedded software implementation provides a custom streamlined Linux Kernel, the necessary kernel-mode drivers, and user-mode application functions suitable to implement the unit. Table 2 in FIG. 6 shows the embedded software architecture and its composition of individual software components. Exemplary embedded system software is also shown in FIG. 6 and/or discussed in the following sections in Table 2.

Application Executive

The Application Executive is a Linux User-mode Process that is launched at boot that runs until the unit is powered off. The purpose of the Application Executive is to serve as a high level state machine that coordinates the various underlying functional components of the system based on user interaction with the unit.

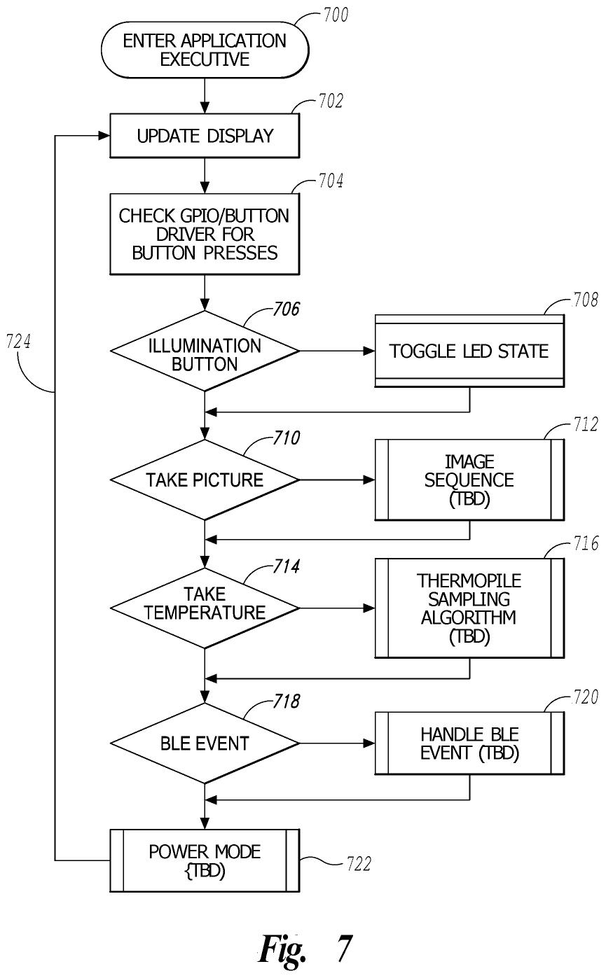

Table 3 in FIG. 7 shows a high level state diagram of the Application Executive 700 which is comprised of a loop 724 and a number of functional components and sub-processes that handles user-events and the various interactions with the hardware components of the system.

The application executive 700 can launch automatically at system boot.

The application executive 700 can start within a desired number of seconds after power-on.

The application executive 700 can run continuously until power-off.

In FIG. 7, application executive 700 is entered, which causes the display to update 702, the a check of the GPIO/Button driver 704. Illumination button 706, take picture button 710, take temperature button 714, and BLE event button 719 are checked for pressings. If a pressing is detected, then, respectively, the following happens: the LED state is toggled 708, the image sequence is initiated 712, the thermopile (or other temperature sensor) sampling algorithm is implemented, and/or the handle BLE event processes are implemented. After such button pressing check 704 is performed (as many iterations as desired), power mode 22 is invoked, which can also lead via loop 724 to update display 702 or other desired location in the loop.

Image Storage

The unit is capable of storing images within its flash file system. Image storage will persist through power cycles. The user of the unit will have the ability to associate a unique patient identifier to a grouping of one or more images. The file system will reside on the same flash part that contains the Linux Kernel and application software; a region of 40 MB is reserved for system software binary storage.

A 40 MB partition of flash can be reserved for Linux Kernel and application software storage.

There can be a Memory Technology Device (MTD) driver suitable to control the eMMC flash interface for use with a Flash File System (FFS)

There can be a FFS implemented.

Image storage can persist through power-cycle.

There can be a unique patient identifier associated with each image.

There can be a method to erase files from the FFS.

Images can be stored using a desired compression algorithm.

Image Capture

The unit is capable of using its camera to capture images for analysis.

There can be a Camera Serial Interface (CSI) driver for image upload from the camera.

There can be an I2C driver for Camera Control Interface (CCI) functionality.

Image data can automatically be written to flash.

Image acquisition sequence can occur automatically when prompted by the user.

Display and Menu

The unit will have a Serial Peripheral Interface (SPI) 128×64 graphical/character. The display will show information pertaining to the current state or function of the unit, as well as host communication status. The display will also be capable of displaying Unique Identifier (UID) information pertaining to the specific unit as well as the current patient. Note: on-device display can be capable or incapable of presenting camera images as desired.

There can be a SPI driver for communications with the display.

The display can be capable of showing current state information.

The display can show a splash screen during system boot.

The display can show the Bluetooth UID of the unit.

The display can show the temperature measurements when prompted by user.

The display can show the current UID of the patient under test.

Temperature Acquisition

The unit is capable of reading a thermal sensor for patient temperature acquisition.

There can be an I2C driver for communication with a thermopile sensor

There can be an algorithm for temperature acquisition.

The unit can acquire temperature when prompted by the user.

There can be a method to associate and store temperature data with the patient UID.

Button Controls

The unit will have three buttons for user interaction. The first button controls the illumination LED (white). The second button initiates the image acquisition procedure. The third button initiates the temperature acquisition procedure. Other buttons can also be provided

There can be a GPIO driver for controlling three button inputs.

There can be a button de-bounce algorithm implemented to filter button noise.

Button-1 can control the state of the illumination LED.

Button-2 can initiate the image acquisition procedure.

Button-3 can initiate the temperature acquisition procedure.

LED Controls

The unit will have three LEDs comprising a white illumination LED, and a red and blue LED used in the image acquisition.

There can be a GPIO driver for controlling three LED outputs.

The white illumination LED output can go active or inactive when prompted by the user.

The red and blue LEDs can be controlled automatically as part of the image acquisition sequence.

Host Communications

Communications with the host PC is achieved through the incorporation of an integrated USB-Bluetooth dongle implementing Bluetooth Low Energy (BLE). Device pairing is performed on the host PC.

There can be a USB-Bluetooth driver and firmware to control the USB-Bluetooth dongle.

After Bluetooth driver registration is complete, the Bluetooth unique identifier can be read and displayed.

The Kernel can include the BlueZ Bluetooth stack.

The unit can present itself as a Basic Imaging Profile (BIP) Bluetooth device if desired.

The unit can transfer images to the host at any desired rate.

Debug Console (Terminal)

The unit will have a serial port used for displaying the Linux Terminal for development and debug.

There can be a UART for serial I/O debug console.

The embedded Linux distribution can include a Terminal console such as bash.

Host Client GUI Software

Graphical User Interface

The host client software can comprise a GUI with minimal functions to utilize the unit. The GUI will have the ability to execute Bluetooth device pairing, file upload and browsing, patient ID display, image display, device wiping, and possibly other functions as desired.

The GUI can be designed to run on the Windows? or 10 Operating Systems.

The GUI can provide an interface for Bluetooth device pairing with one or more units based on the unique Bluetooth device ID.

The GUI can provide an interface to browse the filesystem on the paired unit.

The GUI can provide an interface to upload files from the paired unit to the host PC filesystem.

The GUI can provide the ability to erase files from the paired unit.

The GUI can provide a method of displaying the association of patient unique identifier with patient images and temperature if desired.

The GUI can provide a method of opening and displaying image files.

Turning to some other embodiments and other general discussion, in some embodiments the light path can comprise an illumination light path extending from the scope to the target and the scope can comprise in order a collimator, a 430+/−30 nm notch filter (filter 1), a dichroic filter (filter 2), an unwanted-light absorber, then a glass or other transmissive/transparent window. Such a window can both enhance cleaning and reduce cross-contamination of the device and/or between patients. The illumination light contacts the mucosal tissue or other target tissue then returns through a dichroic filter (filter 2 (the light can pass back past the same dichroic filter), a 475 long pass filter (filter 3), a 590 nm notch filter (filter 4), a filter configured to receive IR and/or NIR light, and then be passed to the detectors and if desired an eyepiece ocular. The filters can be either separate (discrete) or combined (e.g., reflective coatings).

The systems can if desired comprise binocular eyepieces such as loops/filtered glasses or sunglasses/goggles with/without magnification. Some other features that can be included are a light wand, a treatment light, a mirror and/or fiber optic, typically collimated, or an LED on the wand which can have a sleeve with a filter at the end to provide particularly desired light and thus function as the light wand, and thus as the light source or as an additional light source for fluorescence or other desired response.

The scopes' designs can have multi-wavelength light processing within and outside the detector or camera. The light can be piped through the system or a light source can be incorporated or there can be a separate sleeve (or other suitable light emitter) with its own light. The sleeve could have appropriate wavelength emission/excitation filters. Filter and other optical element position can vary within the pathway provided the desired functions are achieved.

The illumination light and viewing pathways can be combined or separate as in a light source with loupes/eyewear. The pathways can enhance user ability to use the device to have a standard method of viewing and illumination. The size of the spot of interrogation in some embodiments is sized to compare a full lesion to surrounding normal tissue, which enhances viewing and identifying anatomical landmarks for location.

In some embodiments, intensity is optimized to bathe the tissue with excitation light for detection and diagnosis, to excite the necessary fluorophores, to induce or avoid heat-based responses, etc. The wavelengths/fluorescence enhance the ability to recognize a shift in the fluorescent emission spectra to permit differentiation between normal and abnormal for cancerous tissue. For example, dual monitoring of two wavelength bands from about 475-585 and from about 595 and up enhances monitoring of cellular activity for the metabolic co-factors NAD and FAD. NAD and FAD produce fluorescence with peak levels at such wavelengths.

In certain embodiments, it is desirable to get as much power as possible without smearing emission signal too much, to keep the output spectrum narrow to prevent Stokes shift, and to exclude UV light and to avoid illuminating/exciting with light in the emission band (overlapping fluorescence).

In certain embodiments, the systems can further comprise a diffuser to make spot-size more regular, remove hot spots, etc. Also sometimes desirable is a collimator to straighten light out at the filter, and to limit the divergence of the beam with increases in power density, or to use a liquid light guide and not fibers so as to get more efficiency by reducing wasted space between fibers, and achieving better transmission per cost and higher numerical aperture (which contributes to better light collection). In still other embodiments, the systems can further comprise metal halide light sources to enhance power in certain emission ranges, dichroic filters or similar optical elements to enhance overlapping viewing and illumination light paths (can simultaneously direct illumination light away from the source and emanation light from the tissue). A glass or other transparent window at the front of the scope can keep out the dust, bodily fluids, infectious organisms, etc. The scopes can be black internally to absorb stray reflected illumination and released fluorescent (unwanted fluorescent feedback) light.

The shape of the scope can be preferably set to be ergonomically comfortable, optimize the excitation and emission pathways. The proximal eyepiece can be set at a length, such that tilting the proximal filter (e.g., a 590 nm notch filter) creates a geometry such that incoming ambient light (if any is relevant) from behind the practitioner can be reduced and what passes can be reflected into the absorbing internal tube surface. This reduces reflection and prevents the user from seeing themselves. For example, the proximal filter can be tilted with its top closer to the clinician and bottom closer to the dichroic mirror so as to make a reflecting surface that would direct incoming light into the bottom of the optical pathway tube.

As noted elsewhere, sometimes multiple light sources can be provided with a single scope. For white light viewing if desired, there could be provision for a greater bandwidth in the output. The larger bandwidth could be obtained by having an extra light (LED, halide, etc.) or by using different filters at the output of a single light source. The systems can also provide illumination with multiple peaks. For example, pharmacology/physiology testing of biological markers may sometimes use this for when fluorescence emitted (by the tissue, markers, or chemical signals) changes in the presence of various ions/molecules/pH. This can also be used to provide a normalization as the power of fluorescence produced by each wavelength can be being compared, normalized against each other.

All terms used herein, are used in accordance with their ordinary meanings unless the context or definition clearly indicates otherwise. Also unless expressly indicated otherwise, the use of “or” includes “and” and vice-versa. Non-limiting terms are not to be construed as limiting unless expressly stated, or the context clearly indicates, otherwise (for example, “including,” “having,” and “comprising” typically indicate “including without limitation”). Singular forms, including in the claims, such as “a,” “an,” and “the” include the plural reference unless expressly stated, or the context clearly indicates, otherwise.

The scope of the present systems and methods, etc., includes both means plus function and step plus function concepts. However, the terms set forth in this application are not to be interpreted in the claims as indicating a “means plus function” relationship unless the word “means” is specifically recited in a claim, and are to be interpreted in the claims as indicating a “means plus function” relationship where the word “means” is specifically recited in a claim. Similarly, the terms set forth in this application are not to be interpreted in method or process claims as indicating a “step plus function” relationship unless the word “step” is specifically recited in the claims, and are to be interpreted in the claims as indicating a “step plus function” relationship where the word “step” is specifically recited in a claim.

The innovations herein include not just the devices, systems, etc., discussed herein but all associated methods including methods of making the systems, making elements of the systems such as particular devices of the scopes, as well as methods of using the devices and systems, such as to interrogate a tissue (or otherwise using the scope to diagnose, treat, etc., a tissue).

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims or other claim having adequate support in the discussion and figures herein.

What is claimed is:

1. A mobile communications housing sized and configured for attachment to a mobile communications device, wherein:
the mobile communications housing comprises a detection system comprising a) an excitation light source configured to emit excitation light selected to elicit fluorescent light from the suspected infection at the target site, b) at least one of a housing camera system configured to selectively accept substantially only fluorescent light emanating from the target site or a camera port sized and configured to selectively transmit substantially only fluorescent light emanating from the target site to an interior camera disposed within the mobile communications device, and c) a heat sensor configured to detect and identify thermal data indicating heat above ambient body temperature emanating from the suspected infection at the target site, and wherein the mobile fluorescence and temperature detector is operably connectable to computer-implemented programming configured to a) accept fluorescent light data associated with the fluorescent light and thermal data associated with heat levels emanating from the target site, and b) interpret the data to determine a probability whether the target site contains an infection.

2. The mobile communications housing of claim 1 wherein the mobile communications housing further comprises a power source operably connected to power at least the excitation light source.

3. The mobile communications housing of claim 1 or 2 wherein the mobile communications housing further comprises a computer containing the computer-implemented programming, and wherein the power source is operably connected to power the computer.

4. The mobile communications housing of claim 3 wherein the computer-implemented programming comprises an app run by a computer disposed within the mobile communications device, wherein the app analyzes the fluorescent light and thermal data to determine the probability whether the target site contains the infection.

5. The mobile communications housing of any one of claims 1 to 3 wherein the excitation light source comprises a light emitting diode configured to emit substantially only the excitation light.

6. The mobile communications housing of claim 5 wherein the excitation light source emits substantially only a single wavelength or wavelength band of excitation light.

7. The mobile communications housing of claim 5 wherein the excitation light source comprises multiple excitation light emitters each emitting a different wavelength or wavelength band of excitation light.

8. The mobile communications housing of claim 5 wherein the excitation light source comprises a white light emitter and at least one short pass filter configured to selectively transmit substantially only light below about 485 nm.

9. The mobile communications housing of claim 5 wherein the excitation light source comprises a light port comprising at least one short pass filter configured to selectively transmit substantially only wavelengths below about 485 nm emitted from a light source disposed within mobile communications device.

10. The mobile communications housing of any one of claims 1 to 9 wherein the mobile communications housing camera system or camera port comprises at least a first long pass filter configured to block the excitation light and a notch filter configured to selectively transmit substantially only fluorescent light emanating from the target area.

11. The mobile communications housing of claim 10 wherein the long pass filter comprises an about 475 nm long pass filter, and the notch filter transmits light have a wavelength of about 590 nm.

12. The mobile communications housing of claim 10 wherein the mobile communications housing camera system or camera port comprises at least one filter configured to selectively transmit substantially only two wavelength bands from about 475-585 nm and at about 595 nm.

13. The mobile communications housing of any one of claims 1 to 12 wherein the mobile communications housing camera system or camera port is configured to selectively accept or transmit, respectively, at least a) substantially only fluorescent light emanating from the target area, or b) all visible light wavelengths emanating from the target area.

14. A mobile communications device configured for detection of a suspected infection at a target site, the mobile communications device comprising a detection system comprising a) an excitation light source configured to emit excitation light selected to elicit fluorescent light from the suspected infection at the target site, b) at least one camera system configured to selectively accept substantially only fluorescent light emanating from the target site, and c) a heat sensor configured to detect and identify thermal data indicating heat above ambient body temperature emanating from the suspected infection at the target site, and,
the mobile communications device further comprises computer-implemented programming configured to a) accept fluorescent light data associated with the fluorescent light and thermal data associated with heat levels above ambient body temperature, and b) interpret the data to determine a probability whether the target site contains an infection.

15. The mobile communications device of claim 14 wherein the mobile communications device further comprises a power source operably connected to power the excitation light source.

16. The mobile communications device of claim 14 wherein the mobile communications device further comprises a computer containing the computer-implemented programming, and wherein the power source is operably connected to power the computer.

17. The mobile communications device of claim 16 wherein the computer-implemented programming comprises an app run by a computer disposed within the mobile communications device, wherein the app analyzes the fluorescent light and thermal data to determine the probability whether the target site contains the infection.

18. The mobile communications device of any one of claims 14 to 16 wherein the excitation light source comprises a light emitting diode configured to emit substantially only the excitation light.

19. The mobile communications device of claim 18 wherein the excitation light source emits substantially only a single wavelength or wavelength band of excitation light.

20. The mobile communications device of claim 18 wherein the excitation light source comprises multiple excitation light emitters each emitting a different wavelength or wavelength band of excitation light.

21. The mobile communications device of claim 18 wherein the excitation light source comprises a white light emitter and at least one short pass filter configured to selectively transmit substantially only light below about 485 nm.

22. The mobile communications device of any one of claims 14 to 21 wherein the camera port comprises at least a first long pass filter configured to block the excitation light and a notch filter configured to selectively transmit substantially only fluorescent light emanating from the target area.

23. The mobile communications device of claim 22 wherein the long pass filter comprises an about 475 nm long pass filter, and the notch filter transmits light have a wavelength of about 590 nm.

24. The mobile communications device of claim 23 wherein the camera port comprises at least one filter configured to selectively transmit substantially only two wavelength bands from about 475-585 nm and at about 595 nm.

25. The mobile communications device of any one of claims 14 to 24 wherein the camera or camera port is configured to selectively accept or transmit, respectively, at least a) substantially only fluorescent light emanating from the target area, or b) all visible light wavelengths emanating from the target area.

26. The mobile communications housing or mobile communications device of any one of claims 1 to 25 wherein the detection system is further configured to determine whether the suspected infection is a viral infection or a non-viral infection.

27. The mobile communications housing or mobile communications device of any one of claims 1 to 26 wherein the camera comprises an imaging system aimed and configured to provide an image of the target site.

28. The mobile communications housing or mobile communications device of claim 27 wherein the image of the target site identifies a spatial organization of the suspected infection.

29. The mobile communications housing or mobile communications device of claim 28 wherein the mobile communications housing utilizes the spatial organization when determining the probability whether the infection is a viral infection or a non-viral infection

30. The mobile communications housing or mobile communications device of any one of claims 1 to 29 wherein, when the suspected infection is a non-viral infection, the computer implemented programming further identifies whether the infection is bacterial.

31. The mobile communications housing or mobile communications device of any one of claims 1 to 30 wherein the at least one light emitter, the light sensor and the heat sensor are all located at a distal end of the mobile communications housing and are all forward-facing and aimed to substantially cover a same area of the target site.

32. The mobile communications housing or mobile communications device of any one of claims 1 to 31 wherein the mobile communications housing is sized and configured to be held in a single hand of a user.

33. The mobile communications housing or mobile communications device of any one of claims 1 to 32 wherein the mobile communications housing is configured to fit within a human oral cavity and to scan at least a rear surface of such oral cavity or a throat behind such oral cavity.

34. The mobile communications housing or mobile communications device of any one of claims 1 to 33 wherein the mobile communications housing further comprises a separable distal element sized and configured to removably attach to the distal end of the mobile communications housing, wherein the separable distal element comprises at least one of light-blocking sides and a forward-facing window configured to selectively transmit at least the excitation light, the fluorescent light and the heat levels without substantial alteration.

35. The mobile communications housing or mobile communications device of claim 34 wherein the separable distal element does not comprise the forward-facing window.

36. The mobile communications housing or mobile communications device of claim 34 wherein the separable distal element comprises both the light-blocking sides and the forward-facing window.

37. The mobile communications housing or mobile communications device of any one of claims 35 to 36 wherein at least two sides of the separable distal element comprise recesses configured to keep the sides out of a view of the heat sensor.

38. The mobile communications housing or mobile communications device of any one of claims 35 to 37 wherein the distal end of the mobile communications housing and the separable distal element are cooperatively configured such that the separable distal element can be snapped on and off the distal end of the mobile communications housing.

39. The mobile communications housing or mobile communications device of any one of claims 35 to 37 wherein the distal end of the mobile communications housing and the separable distal element comprise cooperative projections and detents configured such that the separable distal element can be snapped on and off the distal end of the mobile communications housing.

40. The mobile communications housing or mobile communications device of any one of claims 35 to 37 wherein the distal end of the mobile communications housing is configured to be mounted onto a single circuit board when the mobile communications housing is not being used for scanning.

41. The mobile communications housing or mobile communications device of any one of claims 1 to 40 wherein the mobile communications housing further comprises a display screen on a dorsal side of the mobile communications housing.

42. The mobile communications housing or mobile communications device of any one of claims 1 to 41 wherein the mobile communications housing is configured to account for heat level distortions due to ambient conditions at the target site.

43. The mobile communications housing or mobile communications device of claim 42 wherein the computer-implemented programming further comprises at least one algorithm configured to account for the heat level distortions.

44. A method of scanning an in vivo biological target site for a suspected infection, the method comprising using the mobile communications housing or mobile communications device of any one of claims 1 to 43 to:
emit excitation light selected to elicit fluorescent light from a suspected infection at the target site
sense fluorescent light emanating from the target site elicited by such excitation light;
sense thermal data indicating heat above ambient body temperature emanating from the target site, and
based at least in part on the sensed fluorescent light and the heat levels, determine a probability whether the target site comprises an infection.

45. The method of claim 44 further comprising determining a probability whether the suspected infection is a viral infection or a non-viral infection.

46. The method of claim 45 wherein the method further identifies a spatial organization of the suspected infection.

47. The method of claim 46 wherein the method further utilizes the spatial organization when determining the probability whether the suspected infection is a viral infection or a non-viral infection.

48. The method of any one of claims 44 to 47 wherein, when the suspected infection is a non-viral infection, the method further distinguishes whether the infection is bacterial.

49. The method of any one of claims 44 to 48 wherein the excitation light is emitted by a light emitter located at a distal end of a housing of a hand-held scanning system, and the fluorescent light and the heat levels are detected by sensors located at the distal end of the mobile communications housing or mobile communications device, wherein such light emitter and sensors are all forward-facing and aimed to substantially cover a same area of the target site.

50. The method of claim 49 wherein the mobile communications housing or mobile communications device of configured to be held in a single hand of a user.

51. The method of claim 49 or 50 wherein the mobile communications housing or mobile communications device of configured to fit within a human oral cavity and to scan at least a rear surface of such oral cavity or a throat behind such oral cavity.

52. The method of any one of claims 49 to 51 wherein the system further comprises a separable distal element sized and configured to removably attach to the distal end of the mobile communications housing or mobile communications device, wherein the separable distal element comprises at least one of light-blocking sides and a forward-facing window configured to selectively transmit at least the excitation light, the fluorescent light and the heat levels without substantial alteration, and the method further comprises adding the distal element to and separating the distal element from the mobile communications housing or mobile communications device.

53. The method of claim 52 wherein the separable distal element does not comprise the forward-facing window.

54. The method of claim 52 wherein the separable distal element comprises both the light-blocking sides and the forward-facing window.

55. The method of any one of claims 52 to 54 wherein at least two sides of the separable distal element comprise recesses configured to keep the sides out of a view of the heat sensor.

56. The method of any one of claims 52 to 55 wherein the distal end of the mobile communications housing or mobile communications device of and the separable distal element are cooperatively configured such that the separable distal element can be snapped on and off the distal end of the mobile communications housing or mobile communications device.

57. The method of any one of claims 52 to 55 wherein the distal end of the mobile communications housing or mobile communications device of and the separable distal element comprise cooperative projections and detents configured such that the separable distal element can be snapped on and off the distal end of the mobile communications housing or mobile communications device.

58. The method of any one of claims 52 to 57 wherein the distal end of the mobile communications housing or mobile communications device of configured to be mounted onto a single circuit board when the mobile communications housing or mobile communications device of not being used for scanning.

59. The method of any one of claims 44 to 58 wherein the mobile communications housing or mobile communications device of further comprises a display screen on a dorsal side of the mobile communications housing or mobile communications device.

60. The method of any one of claims 44 to 59 wherein the method further accounts for heat level distortions due to ambient conditions at the target site.

61. The method of any one of claims 44 to 60 wherein the system further comprises at least one algorithm configured to account for heat level distortions due to ambient conditions at the target site.
